(19) United States
(12) Patent Application Publication
(10) Pub. No.: US 2026/0235547 A1
(43) Pub. Date: Aug. 13, 2026

(54) TRIBOELECTRIC NANOSENSOR, DRUG SCREENING PLATFORM, DRUG SCREENING SYSTEM AND DRUG SCREENING METHOD

(71) Applicants: NATIONAL TSING HUA UNIVERSITY, HSINCHU (TW); NATIONAL TAIWAN UNIVERSITY, TAIPEI (TW)

(72) Inventors: Zong-Hong LIN, Taipei (TW); Lee-Wei YANG, Hsinchu (TW); Yi-Yun CHENG, Hsinchu (TW)

(21) Appl. No.: 19/080,949

(22) Filed: Mar. 17, 2025

(30) Foreign Application Priority Data
Feb. 7, 2025 (TW) 114104696

(51) Int. Cl. G01N 27/327; B82Y 15/00; G01N 33/15

(52) U.S. Cl. CPC G01N 27/3278; G01N 27/3276; G01N 33/15; B82Y 15/00

(57) ABSTRACT

A triboelectric nanosensor, a drug screening platform, a drug screening system and a drug screening method are provided. The triboelectric nanosensor includes a single electrode, a solid triboelectric layer, and a fusion protein. The solid triboelectric layer coats a surface of the single electrode. The fusion protein includes a target protein and a histidine tag, and the fusion protein is adsorbed onto a surface of the solid triboelectric layer. The drug screening platform includes the triboelectric nanosensor, a reaction solution, a reaction tank, and a displacement device, in which the reaction solution includes a solvent and a test drug. The drug screening system includes the drug screening platform, a voltage detector and a processor. The drug screening method includes using the drug screening system to perform drug screening.

### RELATED APPLICATIONS

This application claims priority to Taiwan Application Serial Number 114104696, filed Feb. 7, 2025, which is herein incorporated by reference.

### BACKGROUND

### Technical Field

The present disclosure relates to a triboelectric nanosensor and uses thereof. More particularly, the present disclosure relates to a triboelectric nanosensor, a drug screening platform, a drug screening system and a drug screening method.

### Description of Related Art

The emergence of new infectious diseases due to global warming and exacerbated illnesses resulting from lifestyle changes have made the reutilization of existing drugs or the development of new drugs to increase treatment options more urgent than ever. Drug screening is an important step in the modern drug development process, which involves the examination and acquisition of compounds with specific physiological activities. Through standardized experimental methods, compounds with higher activity against a particular target are selected from a large number of compounds or new compounds. With the advancement of drug development technology, experiments on the physiological activity of compounds have gradually transitioned from early verification experiments to screening experiments that conduct horizontal comparisons of the physiological activities of different compounds, which is the so-called drug screening.

In conventional drug screening methods, biochemical assays need to be designed for each individual target protein or time-consuming and concentration-required molecular interaction measurements are required. Conventional high-throughput drug screening methods often produce false-positive results, increasing the time and cost of drug development and leading to the elimination of many initially potentially health beneficial compounds in clinical trials. The average development cost of each drug is estimated to be as high as 2.6 billion USD, with development times exceeding 10 years. Many conventional drug screening methods rely on indirect enzymatic reactions, interpreting the inhibitory effect of the compound on the target protein by the intensity of emitted fluorescence or luminescence signal. However, the measured inhibitory effect may result from inhibition of other enzymes in the reporting system, rather than the target protein. Additionally, drug screening methods such as isothermal titration calorimetry, surface plasmon resonance, and microscale thermophoresis, which measure protein-ligand interactions, are highly sensitive to solvents and impurities contaminated by previous measurements in the equipment and require large amounts of purified proteins. Thus, these drug screening methods cannot be performed in parallel or high-throughput, resulting in low efficiency.

With the development of combinatorial chemistry and computational chemistry, it has become possible to synthesize and separate a large number of compounds in a short period of time. However, pharmaceutical companies design specialized biochemical platforms for each different drug target based on the different biochemical characteristics of each target, which is time-consuming. Additionally, understanding molecular binding through structural biology is expensive and laborious. Currently, there is a lack of universal drug screening platforms. Therefore, developing a drug screening platform and a drug screening method to effectively reduce time and costs of drug research and development is an important issue.

### SUMMARY

According to one embodiment of the present disclosure, a triboelectric nanosensor includes a single electrode, a solid triboelectric layer, and a fusion protein. The solid triboelectric layer coats a surface of the single electrode, and a material of the solid triboelectric layer is nickel oxide. The fusion protein includes a target protein and a histidine tag, wherein the target protein is labeled with the histidine tag, and the fusion protein is adsorbed onto a surface of the solid triboelectric layer.

According to another embodiment of the present disclosure, a drug screening platform includes the aforementioned triboelectric nanosensor, a reaction solution, a reaction tank, and a displacement device. The reaction solution includes a solvent and a test drug. The reaction tank has a reaction space for containing the reaction solution. The displacement device is connected to the triboelectric nanosensor or the reaction tank and configured to periodically and reciprocally contact or separate the triboelectric nanosensor with the reaction solution in the reaction tank to generate a post-reaction output voltage.

According to one another embodiment of the present disclosure, a drug screening system includes the aforementioned drug screening platform, a voltage detector and a processor. The voltage detector is connected to the single electrode and configured to detect the post-reaction output voltage. The processor is electrically connected to the voltage detector and stores a program, and the program performs drug screening when the program is executed by the processor. The program includes a storage module and a calculation module. The storage module stores a pre-reaction output voltage, and the pre-reaction output voltage is generated by the triboelectric nanosensor periodically and reciprocally contacting or separating with the solvent. The calculation module is configured to compare the post-reaction output voltage with the pre-reaction output voltage and calculate an output voltage change to quantify an affinity between the target protein and the test drug.

According to still another embodiment of the present disclosure, a drug screening method includes steps as follows. The aforementioned drug screening system is provided. An electric signal generation step is performed, wherein the displacement device is activated to periodically and reciprocally contact or separate the triboelectric nanosensor with the reaction solution to generate a post-reaction output voltage. A detection step is performed, wherein the voltage detector is used to detect the post-reaction output voltage. A calculation step is performed, wherein the post-reaction output voltage is transmitted to the processor, and the post-reaction output voltage is compared with the pre-reaction output voltage stored in the storage module by the calculation module to calculate an output voltage change to quantify an affinity between the target protein and the test drug.

### BRIEF DESCRIPTION OF THE DRAWINGS

The present disclosure can be more fully understood by reading the following detailed description of the embodiment, with reference made to the accompanying drawings as follows:

FIG. 1 is a schematic view of a triboelectric nanosensor according to one embodiment of the present disclosure.

FIG. 2 is a schematic view of a drug screening platform according to another embodiment of the present disclosure.

FIG. 3 is a schematic view of a drug screening system according to one another embodiment of the present disclosure.

FIG. 4 is a step flow chart of a drug screening method according to still another embodiment of the present disclosure.

FIG. 5A, FIG. 5B and FIG. 5C are AFM images of Comparative Example 1, Example 1 and Example 2, respectively.

FIG. 5D and FIG. 5E are FESEM images of Comparative Example 1 and Example 1, respectively.

FIG. 5F is an EDAX spectrum of Comparative Example 1.

FIG. 5G and FIG. 5H are high-resolution XPS spectra of Comparative Example 1, Example 1 and Example 2.

FIG. 5I shows UPS spectra of Comparative Example 1, Example 1 and Example 2.

FIG. 5J shows analysis results of the root mean square change in roughness of Comparative Example 1, Example 1 and Example 2.

FIG. 5K shows analysis results of the change in work function value of Comparative Example 1, Example 1 and Example 2.

FIG. 6A and FIG. 6B are high-resolution XPS spectra of Comparative Example 2 and Example 3.

FIG. 6C, FIG. 6D, FIG. 6E, FIG. 6F and FIG. 6G show analysis results of concentration-dependent post-reaction output voltage of Example 3.

FIG. 7A and FIG. 7B show analysis results of the relationship between the concentration of the test drug and the post-reaction output voltage after the reaction between the test drug and the triboelectric nanosensor in Example 4.

FIG. 7C and FIG. 7D show analysis results of the relationship between the concentration of the test drug and the post-reaction output voltage after the reaction between the test drug and the triboelectric nanosensor in Comparative Example 3.

FIG. 8A shows the prediction results of the test drug prediction module in the drug screening system of the present disclosure for screening the test drugs.

FIG. 8B is a schematic diagram showing the binding of S130 with Example 2.

FIG. 8C shows analysis results of the relationship between the concentration of S130 and the post-reaction output voltage in Example 5.

FIG. 8D is a schematic diagram showing the binding of tioconazole with Example 2.

FIG. 8E shows analysis results of the relationship between the concentration of tioconazole and the post-reaction output voltage in Example 6.

FIG. 8F is a schematic diagram showing the binding of dexamethasone with Example 2.

FIG. 8G shows analysis results of the relationship between the concentration of dexamethasone and the post-reaction output voltage in Example 7.

FIG. 8H shows results of KPFM analysis surface potential of S130/ATG4B, tioconazole/ATG4B and dexamethasone/ATG4B.

FIG. 8I shows analysis results of the level of ATG4B inhibition in the presence of different drugs.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. Moreover, for the sake of simplicity, some conventional structures and components will be depicted schematically in the drawings and repetitive components can be represented by the same reference numbers.

### Triboelectric Nanosensor

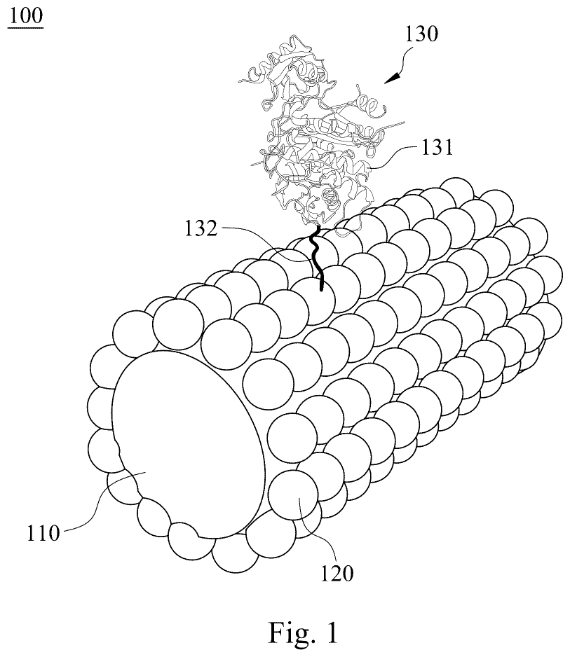

Reference is made to FIG. 1, which is a schematic view of a triboelectric nanosensor 100 according to one embodiment of the present disclosure. The triboelectric nanosensor 100 includes a single electrode 110, a solid triboelectric layer 120, and a fusion protein 130.

The triboelectric nanosensor 100 adopts a single-electrode mode with the earth as a reference electrode, and can collect energy from freely moving objects. Based on two continuous phenomena, contact electrification and electrostatic induction, contact electrification promotes the generation of static and polarized surface charges, leading to electrostatic induction, which then drives the flow of electrons under the potential difference triggered by applied mechanical force, thereby generating an output voltage. Thus, the triboelectric nanosensor 100 is a self-powered chemical sensor that does not require any batteries or external signal conversion circuits during sensing.

The material of the single electrode 110 can be a metal or an alloy, and the metal can be titanium, gold, silver, platinum, aluminum, nickel, copper, tantalum, chromium, selenium, or the alloy thereof. Preferably, the material of the single electrode 110 is copper. The single electrode 110 can be in form of a columnar, a sheet, a strip, a rod, a wire, or a combination thereof.

The solid triboelectric layer 120 coats the surface of the single electrode 110, and a material of the solid triboelectric layer 120 is nickel oxide (NiO). In some embodiments, the solid triboelectric layer 120 surround coats the surface of the single electrode 110. The solid triboelectric layer 120 can use liquid as the contact material, generating electron transfer through the triboelectric effect when the solid triboelectric layer 120 comes into contact with the liquid. Furthermore, the solid triboelectric layer 120 can be a microstructure or a nanostructure, such as but not limited to a nanosheet, a nanoparticle, a nanopowder, a nanofiber, a nanotube, a nanowire, a nanorod, a nanoflower, a nanogroove, a nanopillar, a micropillar, a nanosphere, a microsphere, or a combination thereof.

The fusion protein 130 includes a target protein 131 and a histidine tag 132. The target protein 131 is labeled with the histidine tag 132, and the fusion protein 130 is adsorbed onto a surface of the solid triboelectric layer 120. The target protein 131 refers to a protein of interest for analysis, such as a biomarker in disease progression, an overexpressed protein involved in pathogenic mechanisms, or a receptor and/or a ligand mediating host-pathogen interactions. The histidine tag 132 consists of six or more consecutive histidine residues and can be positioned at either the C-terminus or the N-terminus of the target protein 131. Due to its small size, the histidine tag 132 has minimal impact on the folding structure of the target protein 131. The imidazole functional groups of histidine carry partial negative charges, which can generate an opposite charge attraction with the solid triboelectric layer 120 with a positive charge on the surface. As a result, the fusion protein 130 can be adsorbed onto the surface of the solid triboelectric layer 120 away from the single electrode 110.

### Drug Screening Platform

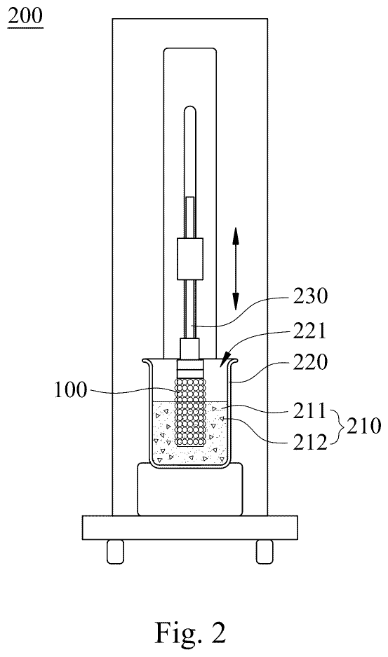

Reference is made to FIG. 2, is a schematic view of a drug screening platform 200 according to another embodiment of the present disclosure. The drug screening platform 200 includes the triboelectric nanosensor 100, a reaction solution 210, a reaction tank 220, and a displacement device 230.

The reaction solution 210 includes a solvent 211 and a test drug 212. The reaction solution 210 can undergo reciprocating motion of contact and separation with the solid triboelectric layer 120 of the triboelectric nanosensor 100, resulting in charge transfer and generating a post-reaction output voltage. Since the liquid has the ability to change shape without the application of force, it can ensure full contact with the surface of the solid triboelectric layer 120 during the contact electrification process, thereby significantly enhancing the output signal. Additionally, the reaction solution 210, as the contact material, can act as a good lubricant and promote smooth motion of the triboelectric nanosensor 100 during the contact and separation operation, thereby increasing the durability of the triboelectric nanosensor 100.

The solvent 211 can be adjusted to a hydrophilic solvent or a hydrophobic solvent depending on the test drug 212, which can be preliminarily determined based on the hydrophilic group or the hydrophobic group in the chemical structure of the test drug 212, or the relevant physicochemical properties of the test drug 212 such as acid dissociation constant (pKa), surface properties, hygroscopicity, solubility in water, etc. Further, the solvent 211 can be water, an organic solvent, or a buffer solution. Water can dissolve most inorganic salts and organic drugs with highly polarity. The organic solvent can be, for example but not limited to, ethanol, propylene glycol, glycerol, polyethylene glycol, dimethyl sulfoxide, dimethyl acetamide, acetone, or a combination thereof. The buffer solution can be, for example but not limited to, phosphate buffered saline (PBS). Additionally, if the test drug 212 is a poorly soluble drug, a solubilizer can be added to the solvent 211 to form a complex, an associate or a double salt between soluble molecules to increase the solubility of the test drug 212 in the solvent 211. The solubilizer can be an organic acid and a sodium salt thereof, such as but not limited to sodium benzoate, sodium salicylate, or para-aminobenzoic acid. The solubilizer can also be an amide compound, such as but not limited to urea, niacinamide or acetamide. Furthermore, if the solvent 211 is highly volatile, its high evaporation rate can lead to rapid evaporation, which facilitates a higher triboelectric output voltage for the triboelectric nanosensor 100.

The reaction tank 220 has a reaction space 221 for containing the reaction solution 210. The reaction tank 220 can be made of a material that does not chemically react with the reaction solution 210 and the single electrode 110 of the triboelectric nanosensor 100, such as glass, polymer, or a combination thereof. In FIG. 2, the reaction tank 220 is shown as cylindrical, but the present disclosure is not limited thereto, and the reaction tank 220 can be in any shape.

The displacement device 230 is connected to the triboelectric nanosensor 100 or the reaction tank 220 and configured to periodically and reciprocally contact or separate the triboelectric nanosensor 100 with the reaction solution 210 in the reaction tank 220 to generate a post-reaction output voltage. The displacement device 230 can be, for example, an oscillator or a dip coater. Through the displacement device 230, the triboelectric nanosensor 100 can actively or passively immerse into or withdraw from the reaction solution 210, enabling the solid triboelectric layer 120 and the reaction solution 210 to undergo periodic reciprocating motion of contact and separation with the reaction solution 210, thereby causing surface charge transfer and generating the post-reaction output voltage.

Furthermore, the drug screening platform 200 can further include a rectifier (not shown), which is connected to the single electrode 110 of the triboelectric nanosensor 100. In some embodiments, the rectifier is connected to the single electrode 110 of the triboelectric nanosensor 100, and the rectifier can further be connected in parallel with a load (not shown). The rectifier can be, for example, a bridge rectifier. The bridge rectifier can be composed of diodes connected in series end-to-end, with one node connected to the single electrode 110 and another node grounded, thereby providing a current path between the induced charge on the single electrode 110 and the ground. The other two nodes of the bridge rectifier are connected to the load of the entire circuit. The potential difference between the single electrode 110 and the ground determines the direction of the current flowing through the bridge rectifier. The drug screening platform 200 can further include a capacitor (not shown), which is connected in parallel with the rectifier and the load. The capacitor can serve as a filter to stabilize the post-reaction output voltage. Additionally, the drug screening platform 200 can further include a resistor (not shown), which is located between the capacitor and the load and is connected in parallel with the capacitor, the rectifier and the load. The resistor can prevent the no-load voltage from being too high due to the action of the capacitor, thereby achieving voltage regulation.

### Drug Screening System

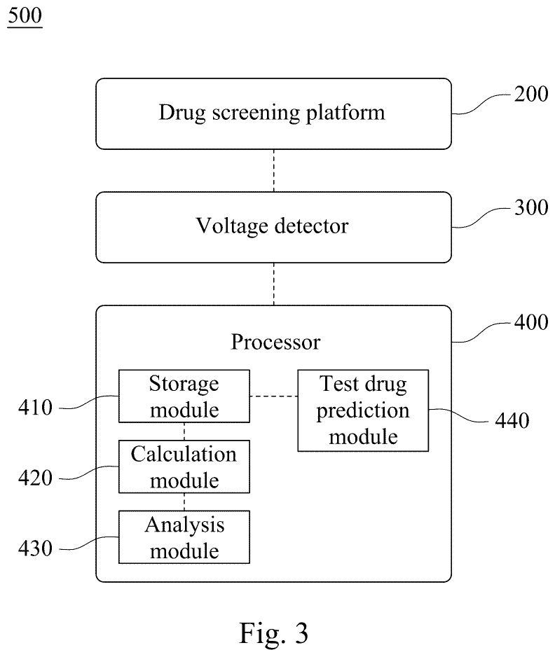

Reference is made to FIG. 3, which is a schematic view of a drug screening system 500 according to one another embodiment of the present disclosure. The drug screening system 500 includes the drug screening platform 200, a voltage detector 300, and a processor 400.

The voltage detector 300 is connected to the single electrode 110 of the triboelectric nanosensor 100 and configured to detect the post-reaction output voltage. The voltage detector 300 can be an instrument capable of detecting voltage changes, such as a voltmeter, a multimeter, an electrochemical analyzer (potentiostat/galvanostat), or an oscilloscope.

The processor 400 is electrically connected to the voltage detector 300 and stores a program (not shown). The program performs drug screening when the program is executed by the processor 400. The program includes a storage module 410 and a calculation module 420. The storage module 410 stores a pre-reaction output voltage, and the pre-reaction output voltage is generated by the triboelectric nanosensor 100 periodically and reciprocally contacting or separating with the solvent 211.

The calculation module 420 is configured to compare the post-reaction output voltage with the pre-reaction output voltage and calculate an output voltage change to quantify an affinity between the target protein 131 and the test drug 212.

The processor 400 can further include a test drug prediction module 440, which is configured to respectively dock a plurality of compounds to the target protein 131, and analyze the binding strength of each of the compounds with the target protein 131 to screen out the test drug 212. The test drug prediction module 440 can be a molecular docking tool, which screens the test drug 212 capable of binding to the target protein 131 from a compound database or newly synthesized drugs. The molecular docking tool includes, but is not limited to, GEMDOCK, DOCK, FlexX, GOLD, ConsDock, AutoDock Vina, GLIDE, ICM, CDOCKER, LibDock, LigandFit and DiffDock, which can be used to perform feature analysis on the binding site of the target protein 131 to obtain the physicochemical properties and relevant information of the binding site. These include, but are not limited to, the types of interactions such as electrostatic force, hydrogen bond, and van der Waals force, the functional groups and residues related to these interactions, the moiety preference of the binding site/binding subsite, as well as the shape and size of the binding site/binding subsite. Based on the analysis of the physicochemical properties of the binding site, compounds with high binding strength to the target protein 131 can be screened as the test drug 212. For example, the dissociation constants (Kd) of the analyzed compounds can be sequentially listed, and the top 10 or top 5 compounds can be selected as the test drug 212 according to the requirements.

Additionally, the processor 400 can further include an analysis module 430 configured to analyze the affinity between the test drug 212 and the target protein 131 to determine whether the test drug 212 is a candidate drug.

### Drug Screening Method

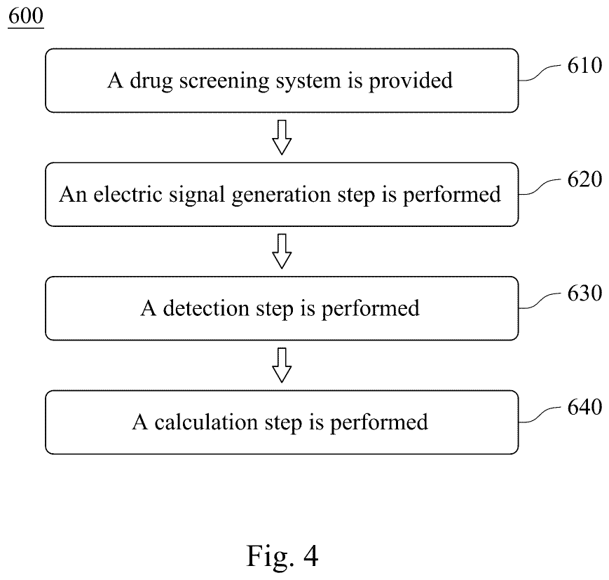

Reference is made to FIG. 4, which is a step flow chart of a drug screening method 600 according to still another embodiment of the present disclosure. The drug screening method 600 includes Step 610, Step 620, Step 630, and Step 640.

In Step 610, the drug screening system 500 is provided. In Step 620, an electric signal generation step is performed, wherein the displacement device 230 of the drug screening platform 200 is activated to periodically and reciprocally contact or separate the triboelectric nanosensor 100 with the reaction solution 210 to generate a post-reaction output voltage.

In Step 630, a detection step is performed. The voltage detector 300 is used to detect the post-reaction output voltage.

In Step 640, a calculation step is performed. The post-reaction output voltage is transmitted to the processor 400, and the post-reaction output voltage is compared with the pre-reaction output voltage stored in the storage module 410 by the calculation module 420 to calculate an output voltage change to quantify an affinity between the target protein 131 and the test drug 212. Specifically, the regression curve of the output voltage change corresponding to different concentration values (approximately 6 to 8 concentration values) of the test drug 212 exhibits an inflection point. The concentration value corresponding to the inflection point can be used to derive the dissociation constant (Kd) of the test drug 212, and the affinity can be estimated based on the Kd of the test drug 212. The reaction of the test drug 212 in the reaction solution 210 and the target protein 131 changes the work function or the hydrophilicity and the hydrophobicity. That is, the work function or the hydrophilicity and the hydrophobicity of the triboelectric nanosensor 100 after the reaction are different from that of the triboelectric nanosensor 100 before the reaction. In some embodiments, the work function of the triboelectric nanosensor 100 after the reaction is lower than that of the triboelectric nanosensor 100 before the reaction. In other embodiments, the work function of the triboelectric nanosensor 100 after the reaction is higher than that of the triboelectric nanosensor 100 before the reaction.

Furthermore, the drug screening method 600 can further include performing a test drug prediction step, where the processor 400 of the drug screening system 500 further includes the test drug prediction module 440. In the test drug prediction step, the test drug prediction module 440 respectively docks a plurality of compounds to the target protein 131, analyzes a binding strength of each of the compounds to the target protein 131, and sequentially lists dissociation constants (Kd) of the compounds to screen out the test drug 212. For example, the top 10 or top 5 compounds can be selected as the test drug 212 according to different target proteins 131 or test requirements, but the present disclosure is not limited thereto. The test drug prediction step involves selecting compounds that may have high binding strength to the target protein 131 from a large number of compounds as the test drug 212, and then further analyzing the affinity between the test drug 212 and the target protein 131 using the drug screening platform 200. The compounds to be analyzed can be selected from a compound database or can be newly synthesized drugs.

The drug screening method 600 can further include performing an analysis step, where the processor 400 of the drug screening system 500 further includes the analysis module 430. In the analysis step, the analysis module 430 analyzes the affinity between the test drug 212 and the target protein 131. When the affinity is higher than a preset threshold, the test drug 212 is determined to be the candidate drug. The preset threshold can be adjusted based on different target proteins 131, for example, using the affinity between the target protein 131 and a known drug as the basis for the preset threshold.

The following specific examples and comparative examples further demonstrate the triboelectric nanosensor, the drug screening platform, the drug screening system, and the drug screening method of the present disclosure. These examples aim to assist person having ordinary skill in the art to fully utilize and practice the present disclosure without excessive interpretation. However, these test examples should not be regarded as limitations of the scope of the present disclosure but are intended to illustrate the materials and methods for implementing the present disclosure.

### 1. The Triboelectric Nanosensor of the Present Disclosure

A triboelectric nanogenerator without adsorbed fusion protein (hereinafter referred to as Comparative Example 1), a triboelectric nanosensor of Example 1 (hereinafter referred to as Example 1), and a triboelectric nanosensor of Example 2 (hereinafter referred to as Example 2) were prepared for testing. In Comparative Example 1, Example 1, and Example 2, a copper (Cu) wire with a diameter of 1 mm was used as the single electrode, respectively. All copper wires were cut into lengths of 3 cm, and were immersed in acetone, isopropanol, and deionized water solution in sequence and placed in a sonicator to remove the impurities from the surface of the single electrode. Prior to sputtering, a length of 1 cm was designated as the sensing area and the rest of the copper wire was masked to prevent the formation of a nickel oxide (NiO) coating. In order to sputter the NiO coating, the copper wire was placed inside the sputtering chamber for 1 hour with the RF power of 100 watts and gas flow of 6 standard cubic centimeter per minute (sccm) of argon. After 1 hour, the NiO coating was successfully sputtered onto the surface of the single electrode as the solid triboelectric layer. Example 1 and Example 2 further adsorbed the fusion protein onto the surface of the solid triboelectric layer. Then the surface morphology of Comparative Example 1, Example 1 and Example 2 were detected using an Atomic Force Microscope (AFM). The target protein in the fusion protein of Example 1 was FKBP, and FKBP was labeled with the histidine tag (hereinafter referred to as His-FKBP). The target protein in the fusion protein of Example 2 was ATG4B, and ATG4B with was labeled the histidine tag (hereinafter referred to as His-ATG4B). During the preparation of the fusion protein, the fusion protein can be isolated and purified using nickel metal chelate affinity chromatography, and then the purified fusion protein can be eluted using a histidine side chain analog imidazole in a concentration gradient and a purity gradient. Specifically, protein solutions (His-FKBP and His-ATG4B) with a concentration of 5×10⁻⁶ M were allowed to react with the solid triboelectric layer at 4° C. for 3 hours. After the reaction, a thin layer of the fusion protein was eventually formed on the surface of the solid triboelectric layer due to the ligand-metal bond formation between Ni and histidine group of the fusion protein. Subsequently, excess protein solution and unbound fusion proteins were washed away using PBS and deionized water to obtain Example 1 and Example 2.

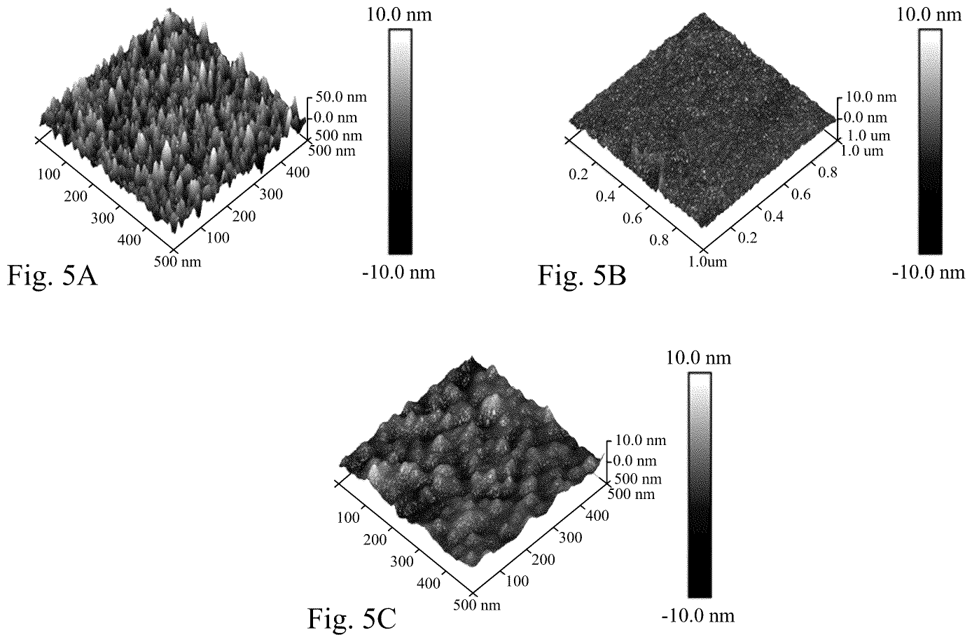

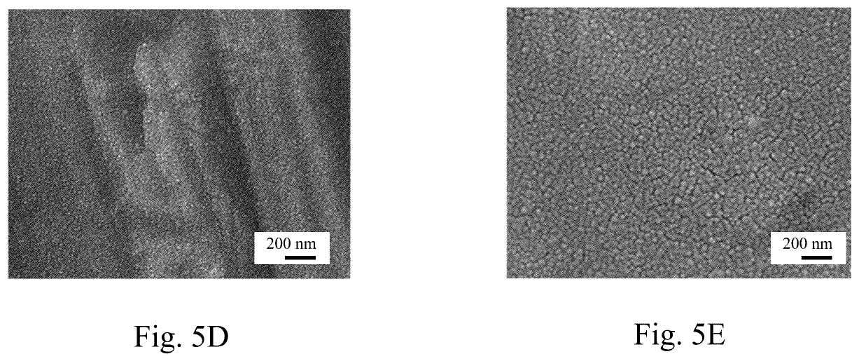

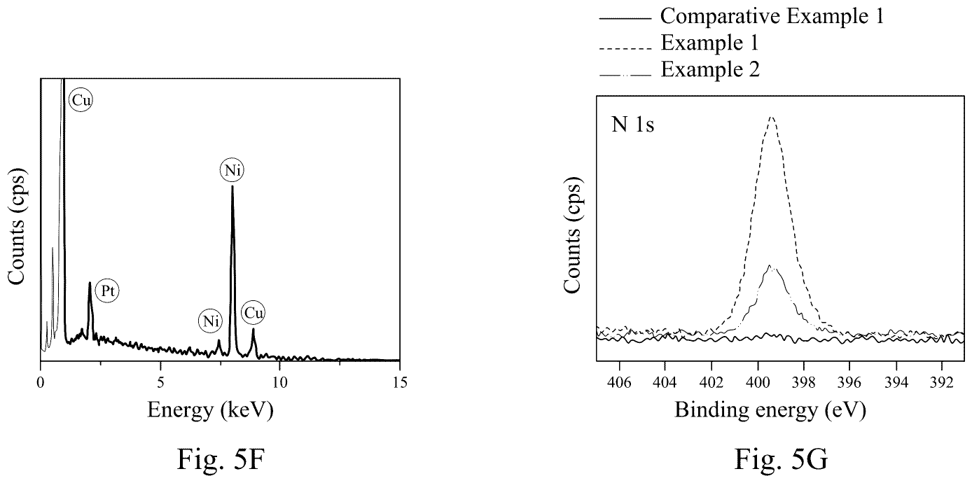

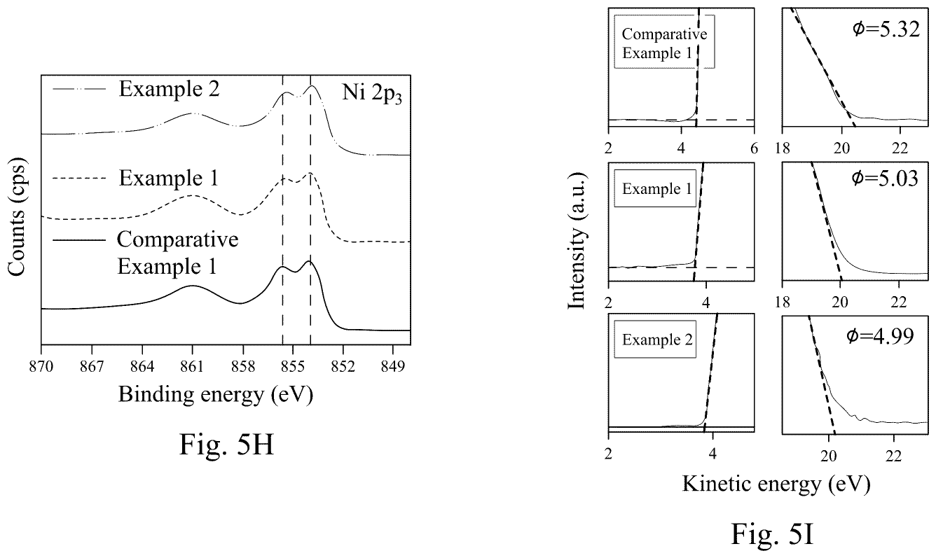

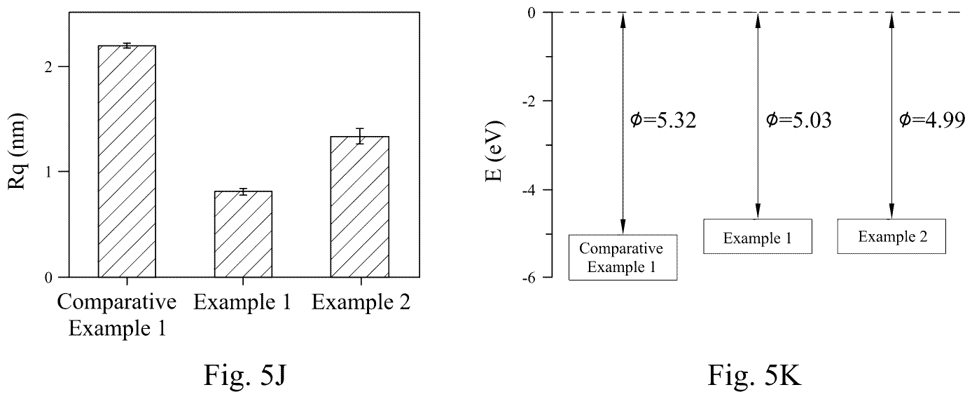

Reference is made to FIG. 5A, FIG. 5B, and FIG. 5C, which are AFM images of Comparative Example 1, Example 1 and Example 2, respectively. In FIG. 5A, the AFM morphology of Comparative Example 1 (NiO coating only) shows many peaks thus indicating surface roughness. However, the results of FIG. 5B and FIG. 5C show that the surface roughness of Example 1 with FKBP adsorbed onto the solid triboelectric layer, and Example 2 with ATG4B adsorbed onto the solid triboelectric layer are both decreased.

The Field Emission Scanning Electron Microscopy (FESEM) was used to detect the surface morphology of Comparative Example 1 and Example 1, and the surface of the single electrode was detected with high-resolution exploration to confirm whether the solid triboelectric layer was successfully formed. Comparative Example 1 and Example 1 were first coated with a layer of platinum (Pt) for 60 seconds at 30 mA to improve the conductivity of the surface. Reference is made to FIG. 5D and FIG. 5E, which are FESEM images of Comparative Example 1 and Example 1, respectively. The results of FIG. 5D show the uniform distribution of NiO on the surface of Comparative Example 1. The average diameter of the as-formed NiO coating on the single electrode was calculated to be 25 nm. As shown in FIG. 5E, the surface uniformity of Example 1 improved after FKBP modification. The Energy Dispersive X-ray Spectroscopy (EDAX) was used to analyze the elemental composition of Comparative Example 1. Reference is made to FIG. 5F, which is an EDAX spectrum of Comparative Example 1. The results of FIG. 5F show the presence of Ni element and Cu element in Comparative Example 1.

Additionally, the X-ray Photoelectron Spectroscopy (XPS) was used to conduct high-resolution XPS analysis to determine the effect of surface modification by FKBP and ATG4B on the Ni 2p core level. Reference is made to FIG. 5G and FIG. 5H, which are high-resolution XPS spectra of Comparative Example 1, Example 1 and Example 2, respectively. As shown in FIG. 5G, the XPS spectrum for Comparative Example 1 did not exhibit any peak for N 1s, whereas the N 1 s peak is observed for Example 1 and Example 2 indicating the presence of FKBP and ATG4B on their surfaces. In FIG. 5H, when measuring binding energy for Ni 2p₃, the peaks were obtained at 853.4 eV, 855.3 eV and 860.9 eV corresponding to the metallic state of Ni, the oxidation state of NiO and the satellite peak of the oxidation state, respectively. The analysis revealed the presence of slightly discernible peak shift, indicating the surface modification induced alterations in the Ni 2p core level. This observation suggests that the modification does not compromise the inherent characteristics of the electrode. Specifically, the absence of significant peak shift at the Ni 2p core level implies that the sensing performance of the triboelectric nanosensor of the present disclosure remains robust and unaltered even after complex surface modifications using the fusion proteins such as FKBP and ATG4B. Therefore, the triboelectric nanosensor of the present disclosure demonstrates reliability and stability, offering the potential for sustained sensing performance and overall effectiveness in anticipated applications.

Furthermore, the Ultraviolet Photoelectron Spectroscopy (UPS) was used to measure the work functions of Comparative Example 1, Example 1, and Example 2. Reference is made to FIG. 5I to FIG. 5K. FIG. 5I shows UPS spectra of Comparative Example 1, Example 1 and Example 2. FIG. 5J shows analysis results of the root mean square change in roughness of Comparative Example 1, Example 1 and Example 2. FIG. 5K shows analysis results of the change in work function value of Comparative Example 1, Example 1 and Example 2.

Based on the UPS spectra in FIG. 5I, the work functions (φ) of Comparative Example 1, Example 1, and Example 2 were calculated from the Fermi level and secondary electron cut-off energy by employing the equation φ=21.22 (E_{Fermi}−ECut _{off}). The calculated work functions for Comparative Example 1, Example 1, and Example 2 are 5.32 eV, 5.03 eV, and 4.99 eV, respectively. It is clear from FIG. 5K that the decrease in work function of Example 1 and Example 2 after modification with the fusion proteins facilitates enhanced charge transfer by overcoming the surface potential barrier. Furthermore, the surface roughness was analyzed after the immobilization of the fusion protein and the root mean square average (Rq) was calculated. The results in FIG. 5J show a significant decrease in the surface root mean roughness of Example 1 and Example 2, consistent with the AFM images in FIG. 5A to FIG. 5C. The results indicate that Comparative Example 1 with the NiO coating has the roughest surface, and roughness decreases upon modification with FKBP and ATG4B in Example 1 and Example 2, respectively. The higher surface roughness of NiO coating provides an increased surface area and potentially more binding sites for the fusion protein to bind on the surface of the NiO coating. Therefore, the triboelectric nanosensor of the present disclosure can serve as a label-free, rapid, and cost-effective sensor for drug screening.

### 2. the Drug Screening Platform of the Present Disclosure

The triboelectric nanosensor of the present disclosure was further applied to a drug screening platform. Firstly, a drug screening platform of Example 3 (hereinafter referred to as Example 3) was constructed to evaluate the sensitivity of the drug screening platform of the present disclosure. In Example 3, the target protein was FKBP, and the test drug in the reaction solution was rapamycin. FKBP is a cytoplasmic protein in the cytosol with high affinity for the immunosuppressant rapamycin. In Example 3, Example 1 was used as the triboelectric nanosensor, and the sensing principle of Example 1 was based on solid-liquid contact electrification. Here, deionized water was used as the solvent, and rapamycin was used as the test drug. The solid triboelectric layer of Example 1 was sequentially contacted with reaction solution containing different concentrations of rapamycin. The experiment also included a drug screening platform of Comparative Example 2, which uses Example 1 as the triboelectric nanosensor and deionized water as the reaction solution (i.e., without rapamycin). The ability of the solid triboelectric layers in Comparative Example 2 and Example 3 to obtain or lose electrons influences the interfacial charge transfer.

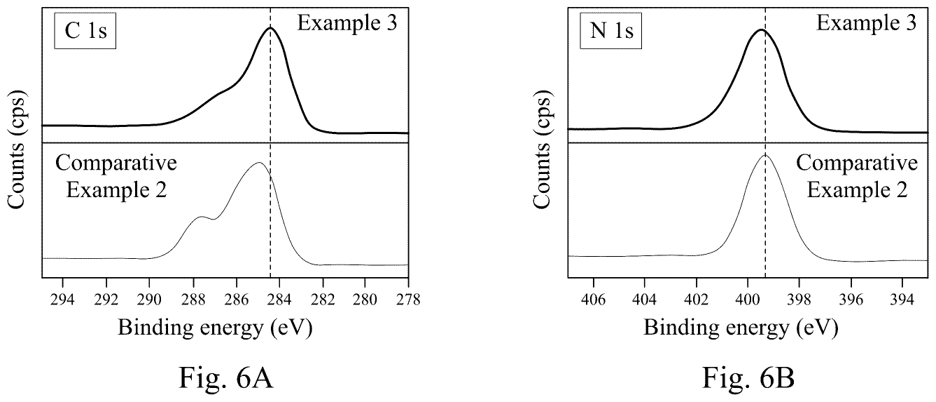

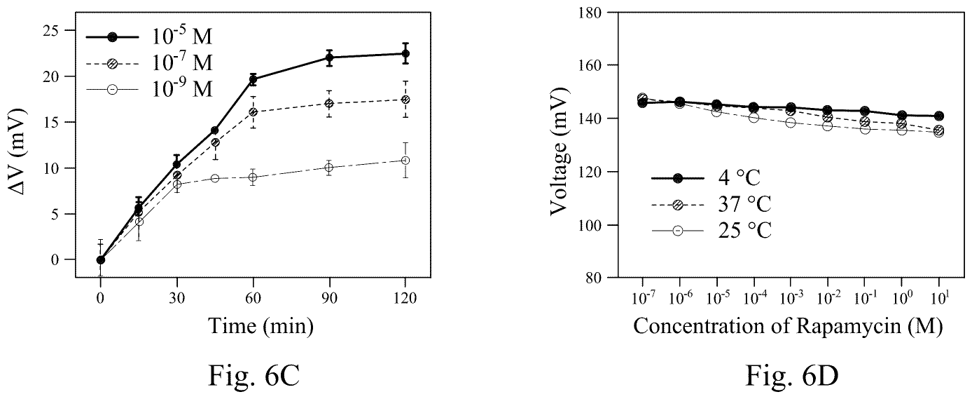

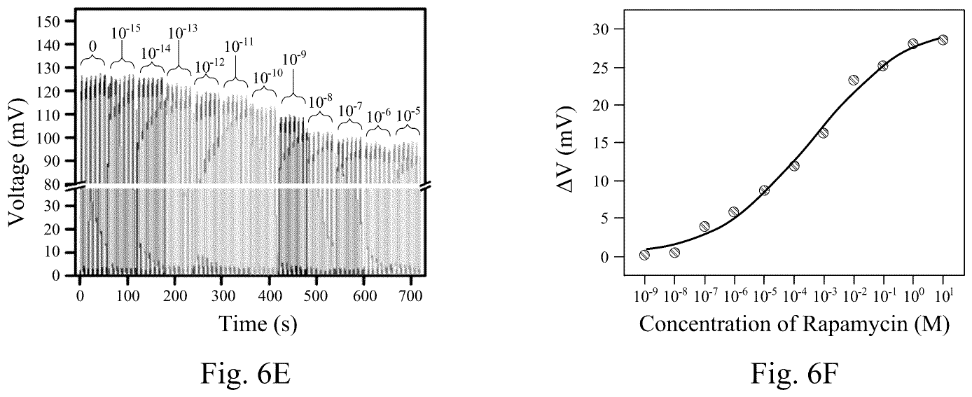

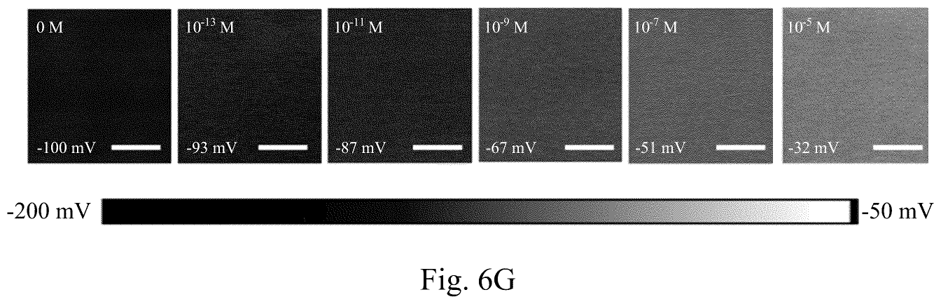

Reference is made to FIG. 6A to FIG. 6G. FIG. 6A and FIG. 6B are high-resolution XPS spectra of Comparative Example 2 and Example 3. FIG. 6C shows the analysis results of concentration-dependent output voltage changes of Example 3 with time. FIG. 6D shows the analysis results of the concentration-dependent post-reaction output voltage of Example 3 at different temperatures and at the first hour. FIG. 6E shows the analysis results of the post-reaction output voltage of Example 3 changing with the concentration of rapamycin. FIG. 6F shows the analysis results of the output voltage changes dependent on the concentration of rapamycin in Example 3. FIG. 6G shows the analysis results of the Kelvin Probe Force Microscope (KPFM) with different concentrations of rapamycin in Example 3.

As shown in FIG. 6A and FIG. 6B, compared to Comparative Example 2, following FKBP modified with rapamycin in Example 3, a noticeable shift in the core energy level spectra of C 1s and N 1s was observed. This shift serves as direct evidence of alterations in the electron density surrounding the carbon (C) atom and the nitrogen (N) atom. This change in electron density is a consequence of the binding with rapamycin, leading to a modified surface with altered electron property. Thus, an output voltage cycle was detected to analyze the sensing activity resulting from the progressive contact electrification and electrostatic induction.

The concentration-dependent output voltage changes of Example 3 were measured using reaction solutions with different concentrations of rapamycin and at different reaction temperatures. Reference is made to FIG. 6C, which measures the output response as a function of reaction time at three different concentrations of rapamycin (10⁻⁵ M, 10⁻⁷ M, and 10⁻⁹ M). The output voltage change results in FIG. 6C show that under the conditions of the concentrations of rapamycin of 10⁻⁵ M, 10⁻⁷ M, and 10⁻⁹ M, the reaction of Example 3 tends to slow down after 1 hour of reaction. Reference is made to FIG. 6D, which shows that the output response at different reaction temperatures (4° C., 25° C., 37° C.) were also observed to be a function of concentrations of rapamycin from 10⁻⁷ M to 10 M. As shown in FIG. 6D, the lower the temperature, the smaller is the response difference found with the increasing ligand (rapamycin) concentration. However, no significant difference was observed between 25° C. and 37° C., indicating that the temperature range of 25° C. to 37° C. is suitable for the reaction.

Based on the above results, a reaction time of 1 hour and room temperature were set as fixed parameters for subsequent experiments. The post-reaction output voltage of Example 3 was measured at different concentrations of rapamycin (10⁻¹⁵ M to 10⁻⁵ M). As shown in FIG. 6E, a steady decrease in output voltage was observed at increasing concentrations of rapamycin. When the concentration of rapamycin was increased from 10⁻¹⁵ M to 10⁻⁵ M, a total reduction of 31 mV in the post-reaction output voltage (126 mV 95 mV) was observed. The surface potential of Example 3 at different concentrations of rapamycin (10⁻¹³ M to 10⁻⁵ M) was measured using the KPFM. As shown in FIG. 6G, a decreasing trend in surface potential values was observed with increasing concentration of rapamycin, which can support the results in FIG. 6E.

Reference is made to FIG. 6F, in which the output voltage shift is plotted as a function of the concentration of rapamycin from 10⁻⁹ M to 10 M. The results in FIG. 6F indicate that the output voltage change increases with the increasing concentration of rapamycin, demonstrating the concentration-dependence of the output voltage change in Example 3. This verifies the sensitivity of rapamycin to the solid triboelectric layer and the fusion protein in the triboelectric nanosensor of Example 1. The sensitivity of the drug screening platform in Example 3 was calculated to be 0.0255 M⁻¹, indicating that the drug screening platform of the present disclosure has the potential for on-site sensing applications. Furthermore, the output characteristics of the drug screening platform were not influenced by changes in the environmental humidity from 30% to 80%, which shows the robustness of the drug screening platform of the present disclosure.

To verify the excellent sensing performance of the drug screening platform of the present disclosure, a drug screening platform of Example 4 (hereinafter referred to as Example 4) and a drug screening platform of Comparative Example 3 (hereinafter referred to as Comparative Example 3) were prepared for testing. In Example 4, the triboelectric nanosensor used a copper (Cu) wire as the single electrode, with a nickel oxide (NiO) coating sputtered onto the surface of the single electrode to serve as the solid triboelectric layer. Then the solid triboelectric layer was reacted with a fusion protein for 2 hours, so that the fusion protein was adsorbed onto the surface of the solid triboelectric layer away from the single electrode. In Example 4, the target protein of the fusion protein is FKBP, which is labeled with the histidine tag. In Comparative Example 3, the triboelectric nanosensor used a copper (Cu) wire as the single electrode, with formed gold nanoparticles (AuNPs) on the surface of the single electrode to serve as the solid triboelectric layer. Then the solid triboelectric layer was reacted with LC-3 for 3 hours, so that LC-3 was adsorbed onto the surface of the solid triboelectric layer away from the single electrode. Subsequently, the drug screening platform of Example 4 was tested with different concentrations of rapamycin, while the drug screening platform of Comparative Example 3 was tested with different concentrations of DK-17.

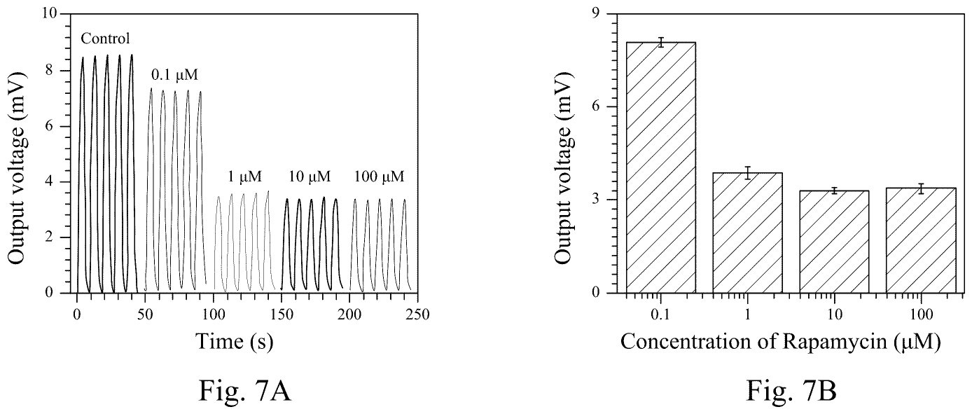

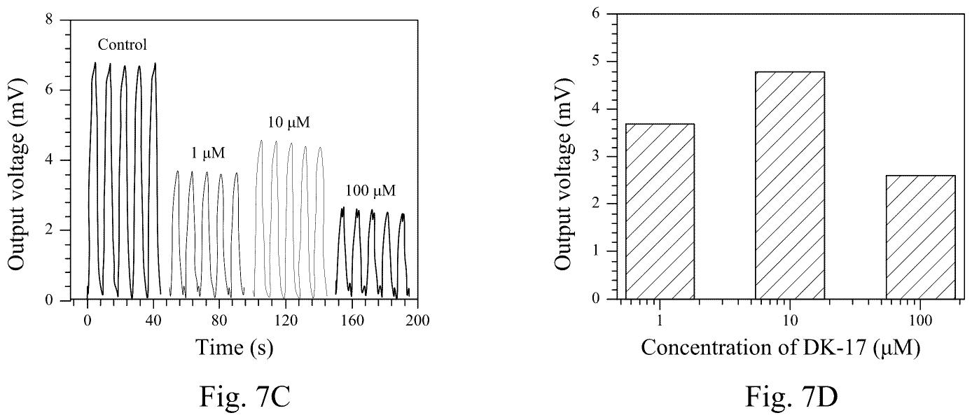

Reference is made to FIG. 7A to FIG. 7D. FIG. 7A and FIG. 7B show analysis results of the relationship between the concentration of the test drug and the post-reaction output voltage after the reaction between the test drug and the triboelectric nanosensor in Example 4. FIG. 7C and FIG. 7D show analysis results of the relationship between the concentration of the test drug and the post-reaction output voltage after the reaction between the test drug and the triboelectric nanosensor in Comparative Example 3. As shown in FIG. 7A and FIG. 7B, the output voltage of the drug screening platform of Example 4 decreased steadily with the increase of the concentration of rapamycin. However, the results of FIG. 7C and FIG. 7D show that the output voltage of the drug screening platform in Comparative Example 3 did not correlate with the concentration of DK-17, indicating that the sensing performance of the drug screening platform in Comparative Example 3 is poor. In contrast, the drug screening platform of the present disclosure exhibits sensitivity and stability.

### 3. The Drug Screening System and the Drug Screening Method of the Present Disclosure

Furthermore, the drug screening platform of the present disclosure was applied to a drug screening system and a drug screening method. Previous research found that ATG4B overexpression can promote the survival of malignant cancers, whereas ATG4B inhibition can significantly enhance tumor sensitivity to chemotherapy in lung cancer, colon cancer, and chronic myeloid leukemia. These findings suggest that ATG4B is a promising anticancer target. Therefore, the triboelectric nanosensor of Example 2 was combined with AutoDock Vina as a test drug prediction module to construct a drug screening system of Example 5, a drug screening system of Example 6, and a drug screening system of Example 7 (hereinafter referred to as Example 5, Example 6, and Example 7). In Example 5, the test drug in the reaction solution was S130. In Example 6, the test drug in the reaction solution was tioconazole. In Example 7, the test drug in the reaction solution was dexamethasone.

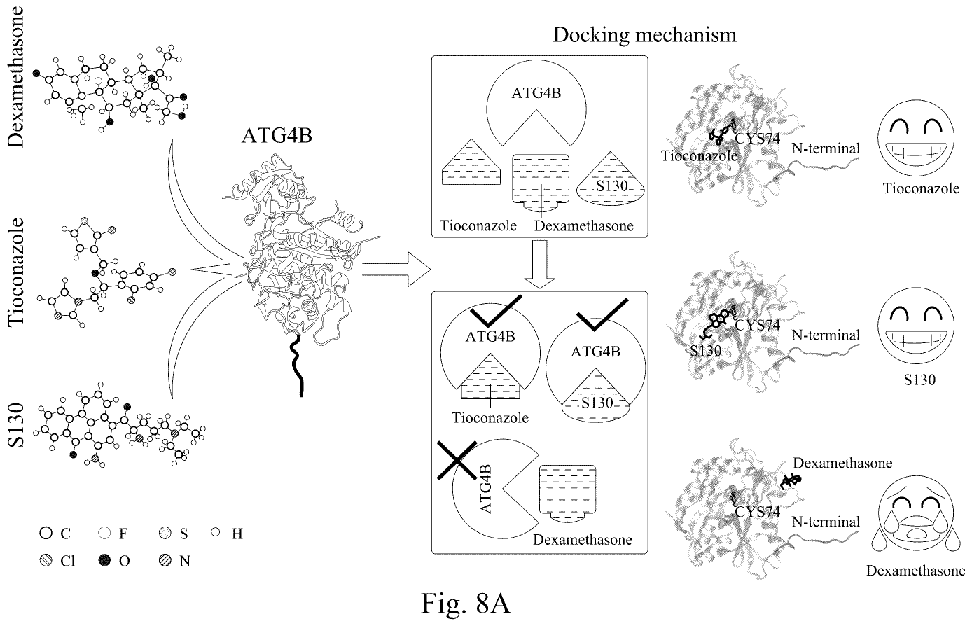

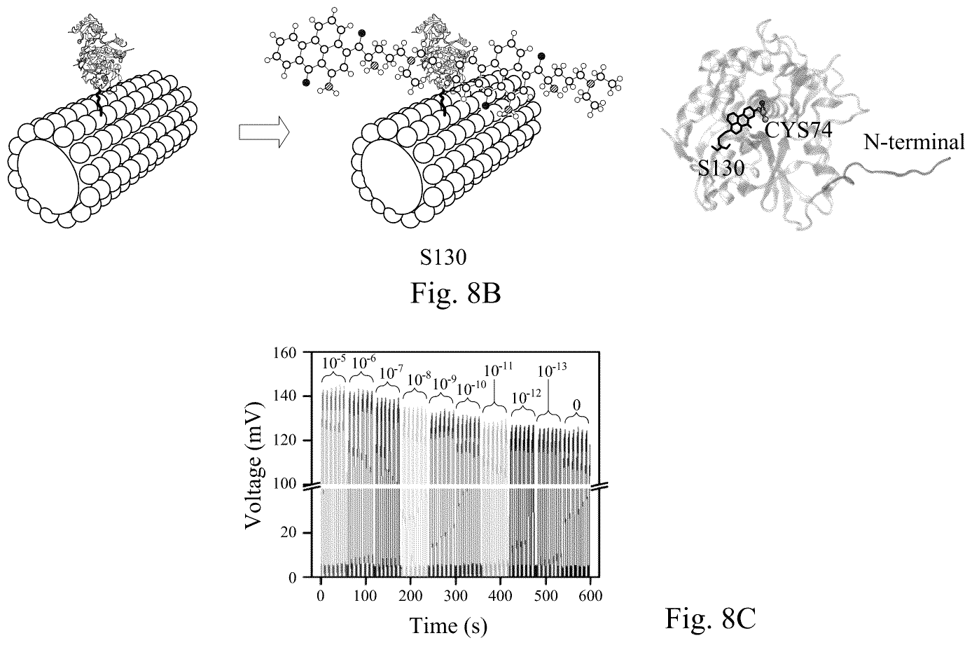

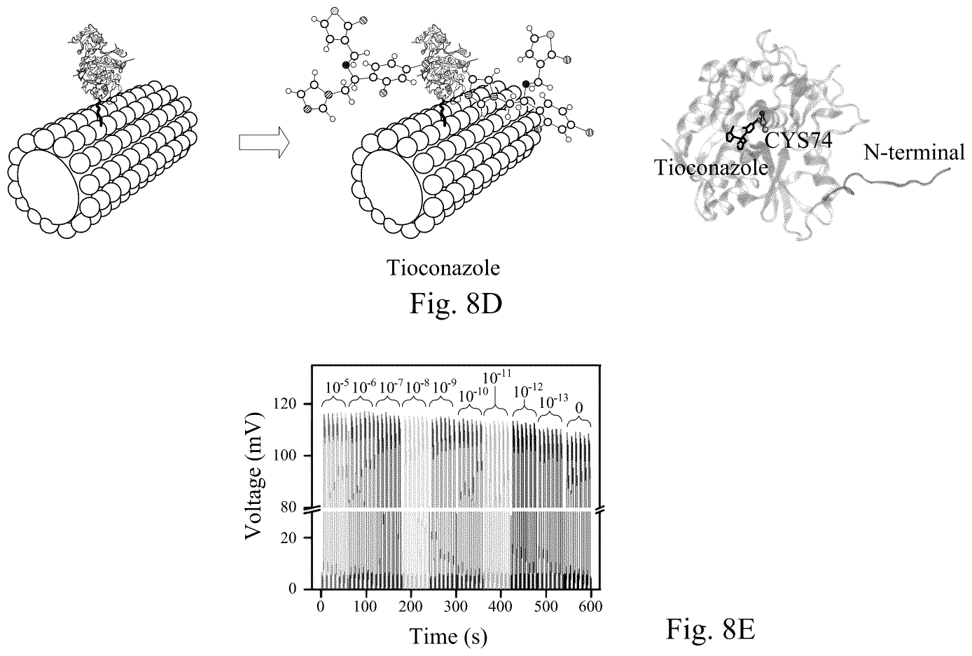

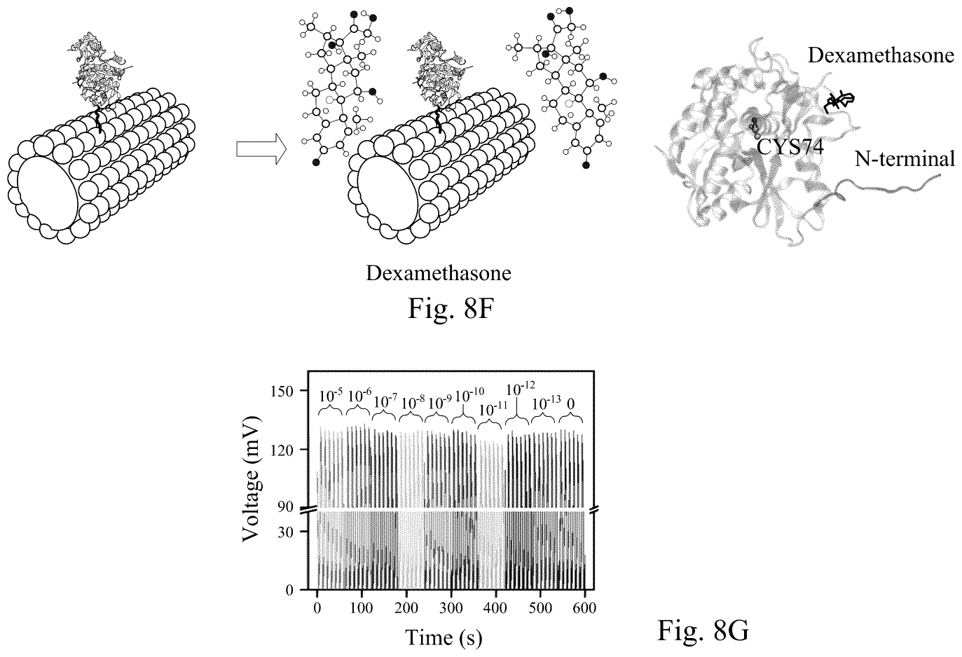

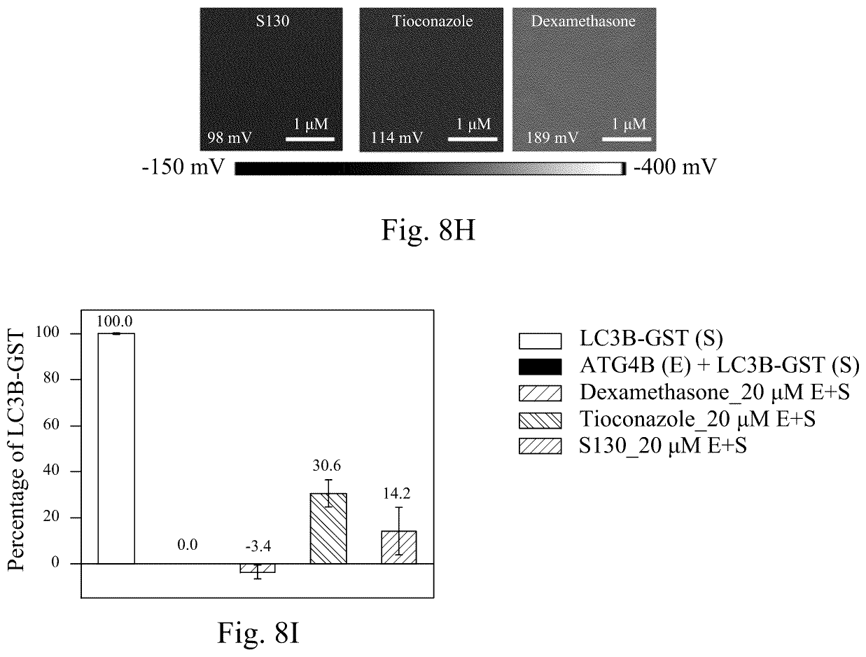

First, AutoDock Vina was used to perform feature analysis on ATG4B (PDB ID: 2Z0D), and the test drugs that could bind to ATG4B were screened from the compound database. Reference is made to FIG. 8A, which shows the prediction results of the test drug prediction module in the drug screening system of the present disclosure for screening the test drugs. In FIG. 8A, ATG4B undergoes small-molecule docking via AutoDock Vina. Among the 20 poses given by the docking results, the lowest-energy pose for tioconazole and S130 were selected. Tioconazole and S130 were previously reported to inhibit ATG4B. As shown in FIG. 8A, S130 is in close proximity to the active site represented by its catalytic cysteine, Cys74. The lowest-energy pose of tioconazole revealed similar results where it can be seen that tioconazole binds directly with the catalytic residue Cys74 of ATG4B in its open/active conformation. Through its dichlorophenyl and chorothiophenyl rings, tioconazole can prevent the C-terminus of LC3 from entering the catalytic site and can contact the active site by hydrophobic contacts and hydrogen bonds. In contrast, among the 20 poses given by the docking results, the lowest-energy pose of dexamethasone was selected. The lowest-energy pose of dexamethasone is distant from the active site represented by its catalytic cysteine, Cys74. These simulations of the test drug prediction module provide preliminary screening results, indicating that tioconazole and S130 are two positive control drugs that bind to active site of ATG4B, while dexamethasone is a negative control known not to bind ATG4B.

Furthermore, the triboelectric nanosensor of Example 2 was respectively tested with different concentrations (0 M, 10⁻¹³ M, 10⁻¹² M, 10⁻¹¹ M, 10⁻¹⁰ M, 10⁻⁹ M, 10⁻⁸ M, 10⁻⁷ M, 10⁻⁶ M, and 10⁻⁵ M) of S130, tioconazole, and dexamethasone to verify the accuracy of the prediction result of the test drug prediction module. Reference is made to FIG. 8B to FIG. 8G. FIG. 8B is a schematic diagram showing the binding of S130 with Example 2. FIG. 8C shows analysis results of the relationship between the concentration of S130 and the post-reaction output voltage in Example 5. FIG. 8D is a schematic diagram showing the binding of tioconazole with Example 2. FIG. 8E shows analysis results of the relationship between the concentration of tioconazole and the post-reaction output voltage in Example 6. FIG. 8F is a schematic diagram showing the binding of dexamethasone with Example 2. FIG. 8G shows analysis results of the relationship between the concentration of dexamethasone and the post-reaction output voltage in Example 7.

The results of FIG. 8C and FIG. 8E show that as the concentration of tioconazole and S130 increased, a positive correlation was seen in the response of output voltage in Example 5 and Example 6, respectively. These results clearly show that the concentration-dependent triboelectric output voltage change corresponding to increasing binding of drug analytes to ATG4B. In contrast, the results in FIG. 8G show that dexamethasone did not show any concentration-dependent change in the output voltage, thus inferring no interaction between dexamethasone and ATG4B.

The change in the triboelectric output voltage can be directly related to the surface potential of the solid triboelectric layer which defines the charge transfer process ensuring the contact electrification. Therefore, to clearly understand the obtained voltage response, the changes in surface potential after binding of different drugs to ATG4B were measured using the KPFM. Lower surface potential indicates decreased potential difference between the solid triboelectric layer and the reaction solution, which in turn generates lower output voltage during the contact and separation operation. Reference is made to FIG. 8H, which shows results of KPFM analysis surface potential of S130/ATG4B, tioconazole/ATG4B and dexamethasone/ATG4B. The results in FIG. 8H show that the surface potentials decrease as the concentration of the reaction solution increases after the reaction of Example 2 with tioconazole and S130.

In parallel, LC3-GST-based enzyme activity assay of purified ATG4B was developed to confirm the inhibitory effects of S130 and tioconazole. ATG4B cleaves the C-terminal fragment of the LC3 precursor protein to produce LC3-I. Therefore, in the presence of the drug, the higher proportion of uncleaved LC3B-GST indicates the better inhibitory effect of the drug on ATG4B. Reference is made to FIG. 8I, which shows analysis results of the level of ATG4B inhibition in the presence of different drugs. The results in FIG. 8I show that 20 μM of S130 suppresses approximately 14% of LC 3-GST cleavage, whereas 20 μM tioconazole inhibits approximately 31% of LC 3-GST cleavage. In contrast, dexamethasone demonstrated no inhibitory effects. These results are consistent with the experimental results of Example 5, Example 6, and Example 7.

In summary, the triboelectric nanosensor of the present disclosure is a label-free and self-powered sensor that does not require markers or external power sources, thereby reducing costs and increasing operational convenience. The drug screening platform and the drug screening system of the present disclosure utilize the rapid response characteristics of the triboelectric nanosensor to quickly detect the affinity between the target protein and the test drug, significantly shortening screening time and accelerating drug development, and quantifying the molecular interactions through output voltage changes, achieving unprecedented selectivity and sensitivity. The drug screening system of the present disclosure has the potential to support high-throughput drug screening for any target protein, with only small amount of the fusion protein required to adsorb onto the surface of the solid triboelectric layer. Thus, the drug screening system of the present disclosure can be applied to drug development for various diseases, offering broad applicability as a high-throughput, low-cost drug screening method. Additionally, the drug screening system and the drug screening method of the present disclosure can be combined with molecular simulation results to examine the same molecular binding event based on different principles. A compound that is determined by both technologies as strongly binding to the target protein is considered the candidate drug, thereby significantly reducing the incidence of false positive. Under the premise that the drug screening platform detects positive interaction, computer technology can efficiently provide the binding site (position) and the binding pose of effective drugs on a large scale. This digital twin approach facilitates the identification of the amino acids that bind to the drug, replacing time-consuming and labor-intensive structural biology experiments that rely on luck to perform site-directed mutagenesis for confirming binding sites. Moreover, the local structural information of the binding sites provided by the computer will guide how the drug can be further chemically modified to enhance its binding capability with the target protein, thereby accelerating the drug development process.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A triboelectric nanosensor, comprising:
a single electrode;
a solid triboelectric layer coating a surface of the single electrode, wherein a material of the solid triboelectric layer is nickel oxide; and
a fusion protein comprising a target protein and a histidine tag, wherein the target protein is labeled with the histidine tag, and the fusion protein is adsorbed onto a surface of the solid triboelectric layer.

2. The triboelectric nanosensor of claim 1, wherein a material of the single electrode is a metal or an alloy.

3. A drug screening platform, comprising:
the triboelectric nanosensor of claim 1;
a reaction solution comprising a solvent and a test drug;
a reaction tank having a reaction space for containing the reaction solution; and
a displacement device connected to the triboelectric nanosensor or the reaction tank and configured to periodically and reciprocally contact or separate the triboelectric nanosensor with the reaction solution in the reaction tank to generate a post-reaction output voltage.

4. The drug screening platform of claim 3, further comprising a rectifier connected to the single electrode of the triboelectric nanosensor.

5. A drug screening system, comprising:
the drug screening platform of claim 3;
a voltage detector connected to the single electrode and configured to detect the post-reaction output voltage; and
a processor electrically connected to the voltage detector and storing a program, wherein the program performs drug screening when the program is executed by the processor, and the program comprises:
a storage module storing a pre-reaction output voltage, wherein the pre-reaction output voltage is generated by the triboelectric nanosensor periodically and reciprocally contacting or separating with the solvent; and
a calculation module configured to compare the post-reaction output voltage with the pre-reaction output voltage and calculate an output voltage change to quantify an affinity between the target protein and the test drug.

6. The drug screening system of claim 5, wherein the processor further comprises a test drug prediction module configured to respectively dock a plurality of compounds to the target protein, and analyze a binding strength of each of the plurality of compounds with the target protein to screen out the test drug.

7. The drug screening system of claim 5, wherein the processor further comprises an analysis module configured to analyze the affinity between the test drug and the target protein to determine whether the test drug is a candidate drug.

8. A drug screening method, comprising:
providing the drug screening system of claim 5;
performing an electric signal generation step, wherein the displacement device is activated to periodically and reciprocally contact or separate the triboelectric nanosensor with the reaction solution to generate a post-reaction output voltage;
performing a detection step, wherein the voltage detector is used to detect the post-reaction output voltage; and
performing a calculation step, wherein the post-reaction output voltage is transmitted to the processor, and the post-reaction output voltage is compared with the pre-reaction output voltage stored in the storage module by the calculation module to calculate an output voltage change to quantify an affinity between the target protein and the test drug.

9. The drug screening method of claim 8, further comprising performing a test drug prediction step, wherein the processor of the drug screening system further comprises a test drug prediction module, and in the test drug prediction step, the test drug prediction module respectively docks a plurality of compounds to the target protein, analyzes a binding strength of each of plurality of the compounds to the target protein, and sequentially lists dissociation constants (Kd) of the compounds to screen out the test drug.

10. The drug screening method of claim 8, further comprising performing an analysis step, wherein the processor of the drug screening system further comprises an analysis module, and in the analysis step, the analysis module analyzes the affinity between the test drug and the target protein, and when the affinity is higher than a preset threshold, the test drug is determined to be a candidate drug.
